(19) United States
(12) Patent Application Publication
(10) Pub. No.: US 2014/0030338 A1
(43) Pub. Date: Jan. 30, 2014

(54) PRODUCT

(71) Applicants: Matthew James Royle, Torfaen (GB); Christina Doyle, Torfaen (GB)

(72) Inventors: Matthew James Royle, Torfaen (GB); Christina Doyle, Torfaen (GB)

(73) Assignee: SPINEART SA, Meyrin (CH)

(21) Appl. No.: 14/002,453

(22) PCT Filed: Mar. 5, 2012
(86) PCT No.: PCT/GB12/50488

§ 371 Date: Oct. 18, 2013

(30) Foreign Application Priority Data
Mar. 3, 2011 (GB) 1103660.5
Mar. 1, 2012 (GB) 1203636.4

(51) Int. Cl. A61K 9/00; A61K 33/42

(52) U.S. Cl. CPC A61K 9/0024; A61K 33/42
USPC 424/489; 424/602

(57) ABSTRACT

A synthetic bone substitute, includes a mixture of osteoconductive particles of first and second average particle sizes, suspended in a water-soluble reverse-phase hydrogel carrier in which the first average particle size is less than about 100 μm, and the second average particle size is about 100-500 μm. A method of producing the same is also described.

### FIELD OF INVENTION

This invention relates to the field of synthetic bone substitutes, and in particular but not exclusively, to synthetic bone substitutes, to methods of producing synthetic bone substitutes, and to methods of using synthetic bone substitutes.

### BACKGROUND TO THE INVENTION

A variety of synthetic bone substitutes are known. The original synthetic bone substitute products were made from either blocks of solid or porous bioactive and osteoconductive materials or comprised bioactive or osteoconductive granules. However, these types of substitutes suffer several disadvantages. They are difficult to fit into uneven spaces in the skeleton when used as solid blocks or may need shaping per-operatively. This can be overcome by using granules, which can be packed into irregular shaped sites. It is difficult to introduce a reproducible volume of material (when used as granules) which will remain cohesive and stay in situ reliably. Granules often need to be pre-mixed with blood or other fluids such as marrow, saline, water, plasma etc., so that they can be more easily handled. Furthermore, granules (even when mixed with coagulated blood) can be washed out of the bone bed by normal blood flow at the site. Even when the granules are mixed with fluid per-operatively, injection of a set dose of bone substitute may be difficult unless a dedicated syringe, through which the particles will flow, is available.

A number of bioactive and osteoconductive materials have been used as synthetic bone substitutes. These include calcium phosphates such as hydroxyapatite, calcium sulphates, bioactive glasses containing silica and calcium ions and variations of these.

One class of synthetic bone substitutes comprises granules of a material such as β-tricalcium phosphate suspended in a reverse phase hydrogel carrier, that is to say a hydrogel which stiffens at body temperature. This stiffening is typically caused by an increase in viscosity. One suitable such hydrogel is a poloxamer. The synthetic bone substitute can therefore be manipulated in use by a surgeon at a temperature of about 10° to 25° C. prior to implantation in a patient's body where it becomes rigid, for example to repair a bone defect. One such synthetic bone substitute is described in US 2006/0110357. This publication discloses a bone putty composition comprising tricalcium phosphate or other calcium phosphate granules suspended in a carrier formulation including a reverse phase poloxamer hydrogel. The publication discloses the use of granules of tricalcium phosphate with a size range of from about 100 μm to about 425 μm.

A significant problem with known synthetic bone substitutes based on a hydrogel is that typical sterilisation methods, i.e. gamma irradiation and electron beam sterilisation, can cause cross-linking of the hydrogel's polymers which modifies its viscosity and causes stiffening. The necessary sterilisation process therefore affects the handling characteristics of the synthetic bone substitute. US 2006/0110357 indicates that electron beam irradiation can be used to increase the molecular weight of a poloxamer carrier used in a synthetic bone substitute to increase the viscosity of the bone substitute at cold temperatures which might be experienced after sterilisation, for example during shipping. Specific increases in the molecular weight of the poloxamer carrier substance are suggested.

Whilst it is possible to control irradiation to achieve sterilisation, it is well known that polymeric materials may be altered by the energy added to the material during radiation. As suggested above, a number of events can potentially be induced by radiation. For example, bonds in the material can crosslink and make the material stiffer and brittle, the bonds can be broken and the molecular weight reduced (reducing stiffness and strength) or the material may suffer from long term degradation if oxygen free radicals are generated. Consequently care must be taken in discovering how a polymer behaves and testing its properties post-irradiation, i.e. as it is used by the surgeon.

US2009/0143830 discloses another synthetic bone substitute composition based on a reverse phase carrier and an alloplastic material which can be hydroxyapatite or a calcium phosphate including β-tricalcium phosphate. Different compositions are disclosed in this publication, from a paste-like form comprising about 50% by weight of the alloplastic material and about 50% by weight of the reverse phase carrier; to a gel-like composition comprising about 40% by weight of the alloplastic material and about 60% by weight of the carrier. The alloplastic material particles are said to have a mean length of about 0.08-5.0 mm (80-5000 μm) and a maximum diameter of about 2.0 mm (2000 μm).

U.S. Pat. No. 6,949,251 discloses a porous β-tricalcium phosphate material for bone implantation formed by β-tricalcium phosphate granules. The size of the granules is in the range 250-1700 μm, preferably 1000-1700 μm, most preferably 500-1000 μm.

US2004/0022858A discloses a synthetic bone substitute composition comprising demineralised bone powder and a reverse phase carrier such as a poloxamer. The bone powder is provided in particles having a mean length of 0.25-1 mm (250-1000 μm) and a mean thickness of about 0.5 mm (500 μm).

Although synthetic bone substitute compositions have been used clinically clinicians still complain that the substitutes do not readily flow and are not easy to manipulate. Furthermore, care must be taken that the substitute is not washed out of the defect shortly after implantation by the action of blood and other fluids.

An object of the present invention is to provide a synthetic bone substitute having improved handling characteristics. Preferably the synthetic bone substitute is malleable, enabling it to be manipulated by a surgeon to pack material into a bone defect, and also so it can be injected into the site being treated directly from, for example, a syringe. In particular, it is an object of the invention to provide a synthetic bone substitute which is both malleable and capable of being injected from a syringe.

Another object of the invention is to provide a synthetic bone substitute which can remain malleable after sterilisation. A further object of the invention is to provide a simplified manufacturing process for a synthetic bone substitute.

### SUMMARY OF THE INVENTION

According to one aspect of the invention there is provided a synthetic bone substitute, comprising a mixture of osteoconductive particles of first and second average particle sizes, suspended in a 30 to 40% weight for weight concentration of a water-soluble reverse-phase hydrogel carrier, in which the first average particle size is less than about 250 μm and the second average particle size is about 250-500 μm. In a preferred embodiment of the invention there is provided a synthetic bone substitute, comprising a mixture of osetoconductive particles of first and second average particle sizes, suspended in a water-soluble reverse-phase hydrogel carrier, in which the first average particle size is less than about 100 μm and the second average particle size is about 100-500 μm.

The synthetic bone substitute of the invention is advantageous in that it has improved handling properties compared to known synthetic bone substitutes, remaining malleable even after sterilisation. The improved handling properties are achieved without the problems associated with sterilisation seen in the synthetic bone substitutes of the prior art.

The broad range of particle sizes facilitates rapid vascularisation of the graft site providing for an infusion of bone-forming cells, enhancing the processes of new bone development and resorption of the scaffold. The body responds to the particles in a similar way to its response to normal extracellular bone mineral.

The particles preferably have a mean particle size of around 300 to 400 μm, preferably between 325 and 375 μm, especially between 335 and 360 μm. In embodiments of the invention, the particles have a mean particle size of about 150 to 500 μm, preferably between 200 and 500 μm, more preferably between 250 and 400 μm.

The synthetic bone substitute of the invention can comprise particles having a particle size distribution within the range d10=<20 μm, d50=<400 μm and d90=<500 μm, more preferably within the range d10=<15 μm, d50=<350 μm, and d90=<450 μm and in a particular embodiment of the invention, the particle size distribution is within the range d10=<10 μm, d50=<300 μm and d90=<400 μm. In a preferred embodiment of the invention d5=<10 μm, d30=<200 μm, d90=<600 μm and d99=<700 μm, preferably d5=<5 μm, d30=<100 μm, d90=<500 μm and d99=<600 μm and in a particular embodiment of the invention d5=5 μm, d30=100 μm, d90=500 μm and d99=600 μm.

Particle size preferably refers to the length of the longest dimension of the particles. Other dimensions can be used, but it is preferable that all the particles in one substitute are measured using the same dimension. Particle size and/or distribution can be measured using known laser diffraction particle size analyzers, such as an LS particle size analyzer available from Beckman Coulter®.

The shape of the particles may be selected so as to achieve improved flow of the synthetic bone substitute and also to improve bone interaction. It is preferred that the particles are not spherical. In particular, the particles preferably have an aspect ratio (the ratio of the particle width to length) of 1:X, wherein X is greater than 1, especially approximately or greater than 1.2, 1.5, 1.8, 2, 3 or 4.

The first average particle size is less than about 250 μm. Particles having a first average particle size preferably have a particle size between 50 and 300 μm, more preferably between 100 and 250 μm, more preferably between 150 and 250 μm, even more preferably between 175 and 225 μm. In embodiments of the invention, particles having a first average particle size can have a particle size of less than 100 μm, preferably between 1 and 100 μm, more preferably between 1 and 50 μm, even more preferably between 3 and 30 μm, and more preferably still, between 4 and 20 μm. The largest particles having the first average particle size are preferably no more than 100, 75, 50 or 25 μm larger than the smallest particles having the first average particle size.

The second average particle size is between about 250 μm and 500 μm. Particles having a second average particle size preferably have a particle size between 250 and 600 μm, more preferably between 300 and 500 μm, more preferably between 350 and 450 μm. In embodiments of the invention, particles having a second average particle size can have a particle size between 100 and 500 μm, preferably between 125 and 450 μm, more preferably between 150 and 450 μm, even more preferably between 175 and 425 μm. The largest particles having the second average particle size are preferably no more than 100, 75, 50 or 25 μm larger than the smallest particles having the second average particle size. In embodiments of the invention the largest particles having the second average particle size are preferably no more than 300, 250, 200 or 150 μm larger than the smallest particles having the second average particle size.

The first average particle size is preferably around or less than 150, 100, 75, or 50 μm smaller than the second average particle size. In embodiments of the invention the first average particle size is preferably around or less than 500, 400, 300 or 200 μm smaller than the second average particle size.

The synthetic bone substitute may additionally include particles having a third average particle size. The third average particle size is between about 250 μm and 400 μm. Particles having a third average particle size preferably have a particle size between 250 and 400 μm, more preferably between 250 and 350 μm, more preferably between 275 and 325 μm. The largest particles having the third average particle size are preferably no more than 100, 75, 50 or 25 μm larger than the smallest particles having the third average particle size.

The first average particle size is preferably around or less than 150, 100, 75, 50 or 25 μm smaller than the third average particle size.

The osteoconductive particle may be a particle of any appropriate material such as a ceramic or glass. Such materials are known for use in this field and include tricalcium phosphate (especially β-tricalcium phosphate), hydroxyapatite, calcium sulphate and bioactive glass. Preferably the material is β-tricalcium phosphate. Tri-calcium phosphate is a calcium phosphate mineral with a calcium to phosphate ratio of about 1.5 (compared with a calcium to phosphate ratio of 1.67 for hydroxyapatite). It is more rapidly resorbed in the body than hydroxyapatite.

Average particle size may be controlled physically, for example by sieving the particles, and determined, for example by scanning electron micrograph analysis. Optionally, the osteoconductive particles can be sintered to a particular hardness before and/or after sieving. The particles may also be subjected to grinding, and combinations of one or more of sintering, sieving and grinding may be used to control particle size.

The hydrogel is preferably a poloxamer, which is a high molecular weight hydrogel. Poloxamers are nonionic triblock copolymers composed of a central hydrophobic chain of polypropylene oxide flanked by two hydrophilic chains of polyethylene oxide. Suitable poloxamers include a block polymer of polypropylene oxide and ethylene oxide, the formula of which is provided below as formula 1;

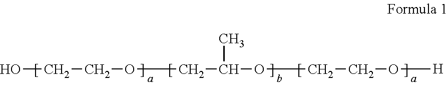

wherein a and b are independently integers between X and Y. It is particularly preferred that a is greater than b, especially at least 10% greater, 20% greater, 30% greater, 50% greater, 75% greater or 90% greater. It is particularly preferred that the value of b is between 30 and 60% of the value of a, more preferably between 40 and 60% of the value of a. In one embodiment, a is between 80 and 120, more preferably between 90 and 110, even more preferably between 95 and 105. It is especially 100, 101, 102, 103, or 104. In the same or another embodiment, b is preferably between 35 and 70, more preferably between 40 and 60, especially between 50 and 60, especially 54, 55, 56 or 57. When a is 101, b is preferably 56.

The advantage of using a poloxamer which is reverse phase, that is to say it stiffens as the temperature rises, is that it is less likely to flow away at body temperature, unlike conventional carriers or binders which can drain away easily when injected. The poloxamers that can be used in the current invention do not drain away as easily and so will remain in place whilst the bone substitute is introduced into the site at which it is required. The poloxamer will then gradually dissolve away on contact with body fluid.

The dissolution process of the gel leaves a three-dimensional scaffold with interconnected pores that mimics the geometry of human cancellous bone matrix in-situ in the defect.

A suitable hydrogel for use in the synthetic bone substitute of the present invention may comprise about 10% to about 50% weight for weight concentration of poloxamer beads, preferably about 20% to about 40%, more preferably about 30%. The hydrogel may additionally comprise about 50% to about 90% weight for weight concentration of water, preferably about 60% to about 80%, more preferably about 70%.

In one embodiment, the synthetic bone substitute comprises about 30% weight for weight concentration of the hydrogel carrier, especially between 28 and 33%. In another embodiment, the synthetic bone substitute comprises about 40% weight for weight concentration of the poloxamer carrier, especially between about 38 and 43%. This embodiment is particularly suitable for use in conjunction with implants, such as posterior lumbar interbody cage fusion devices.

In an alternative embodiment of the invention, the synthetic bone substitute may comprise about 20% to about 70% by volume of the hydrogel carrier, preferably about 30% to about 50% and more preferably about 40%. The synthetic bone substitute may additionally comprise about 30% to about 80% by volume of the osteoconductive particles, preferably about 40% to about 70%, more preferably about 60%.

Adjusting the concentration of the hydrogel prior to irradiation has a direct correlation to the handling characteristics achievable in the post-irradiated synthetic bone substitute. The ratio of osteoconductive particles to hydrogel has been observed to affect extrusion and handling characteristics of the synthetic bone substitute.

The synthetic bone substitute may also include other components such as a radio-opaque material; or a component which increases the visibility of the synthetic bone substitute in use so that it can be visibly distinguished by a surgeon from natural bone. The synthetic bone substitute may include other components such as bone powder, whether mineralised or demineralised, a growth factor or a bone morphogenic protein, such as BMP 7 or BMP 2. Optionally, it can include autologous, allograft or xenograft bone. It may also include bone marrow, especially bone marrow harvested from the individual to which the substitute is to be administered. Further materials may include gypsum, hydroxyapatites, another calcium phosphate, calcium carbonate or calcium sulphate, bioactive glass and any other biocompatible ceramic and combinations of these components.

Preferably the synthetic bone substitute of the invention has a complex modulus plateau of more than 3×10³ Pa at 10° C. and a complex modulus plateau of less than 3×10⁶ Pa at 37° C. The synthetic bone substitute of the invention preferably has a complex modulus plateau of greater than 8×10⁵ Pa at 20° C. The synthetic bone substitute of the invention may have an interpolated yield stress of less than 50 Pa at 10° C. and an interpolated yield stress of greater than 4000 Pa at 37° C. The synthetic bone substitute of the invention preferably has an interpolated yield stress of greater than 1000 Pa at 20° C. Preferably it has a zero stress viscosity of between 4.5×10⁷ Pa·s and 6×10⁷ Pa·s, more preferably between 4.75×10⁷ Pa·s and 5.75×10⁷ Pa·s, especially between 4.8×10⁷ Pa·s and 5.6×10⁷ Pa·s.

The surface of the particles is preferably rough. This may be created by roughening the surface. A rough surface may be provided in one embodiment by pores in the particles. When the particles are porous, the pores may be any size, but are preferably between 1 μm and 200 μm in diameter, more preferably between 50 μm and 150 μm.

The density of the particles may be varied by varying the porosity and the pore size. For example, the particles may be between 30% and 85% porous, more preferably between 40% and 80% porous, more preferably between 40% and 60% or 60% and 80% porous. The porosity may be selected according to the strength of the particle material, a stronger material allowing a more porous structure.

The synthetic bone substitute of the present invention is preferably porous, this porosity being created due to the higher density osteoconductive particles being suspended in resorbable, lower density hydrogel phase. The greater resorption rate of the hydrogel matrix results in assimilation of the gel, where cells penetrate macroporous gaps present between particles, leaving a network of osteoconductive particles to facilitate rapid neovascularisation. The size of the hydrogel struts separating the particles is generally controlled by the particle size distribution. In the present invention the percentage volume porosity of the synthetic bone substitute is ideally the same as the ratio of the hydrogel:particles, being about 20% to about 70% by volume, preferably about 30% to about 50% and more preferably about 40%.

Porosity can be measured using known X-ray microtomography (micro-CT) instruments such those supplied by SkyScan™.

According to another aspect of the invention there is provided a kit comprising packaging and/or a delivery device, and synthetic bone substitute in accordance with the invention. The packaging and/or delivery device is preferably sterile. The packaging or delivery device may be in the form of single use or multiple use configurations.

The delivery device may be, for example, a syringe which is loaded with synthetic bone substitute, and which is suitable for use in administering the synthetic bone substitute to repair a bone defect or to fill an implant.

According to another aspect of the invention there is provided a method of producing a synthetic bone substitute, the method comprising providing a mixture of osteoconductive particles of first and second average particle sizes, in which the first average particle size is less than about 250 μm and the second average particle size is about 250-500 μm, and suspending the particles in a hydrogel, preferably a poloxamer, carrier. The invention also provides a method of producing a synthetic bone substitute, the method comprising providing a mixture of osteoconductive particles of first and second average particle sizes, in which the first average particle size is less than about 100 μm and the second average particle size is about 100 to 500 μm. Various techniques are known for providing populations of granules having different average particle sizes. One preferred technique is to sieve a mixture of β-tricalcium phosphate granules.

The particles and carrier are preferably as defined in relation to the first aspect of the invention.

Preferably the mixture of β-tricalcium phosphate particles and poloxamer hydrogel carrier comprises about 30-40% by weight poloxamer carrier. Preferably the concentration of poloxamer carrier is 28-32%, more preferably 29-31%, most preferably about 30%. In a preferred embodiment of the invention the mixture of β-tricalcium phosphate particles and poloxamer hydrogel carrier comprises about 30-50% by volume hydrogel carrier, preferably 35-45%, most preferably about 40%.

According to another aspect of the invention there is provided a synthetic bone implant comprising a synthetic bone substitute according to the invention. The implant may be shaped to fill a bone defect.

According to a further aspect of the invention there is provided a method of repairing a bone defect, the method comprising introducing a synthetic bone substitute according to the invention into the bone defect and allowing the synthetic bone substitute to set.

The bone defect may be naturally occurring, for example as a result of injury such as a fracture, or artificially generated—such as an insertion hole for a bone screw.

Also provided is the synthetic bone substitute according to the first aspect of the invention for use in therapy, particularly for use in the treatment or repair of a bone defect. The synthetic bone substitute of the present invention may also be used to assist bone healing (e.g. in spinal fusion) or to repair gaps caused during the failure of primary joint replacements.

The synthetic bone substitute according to the invention is particularly suitable for use in arthroscopic or endoscopic procedures, because of its injectability and radio-opacity. It is also useful in dental procedures.

### BRIEF DESCRIPTION OF THE DRAWINGS

A synthetic bone substitute in accordance with the invention, and methods for its preparation and use, will now be described, by way of example only, with reference to the accompanying drawings, FIGS. 1 to 10 in which:

FIG. 1 is a scanning electron micrograph of β-tricalcium phosphate particles used in a synthetic bone substitute in accordance with the invention;

FIG. 2 is a scanning electron micrograph of particles of β-tricalcium phosphate from an existing synthetic bone substitute (Actifuse®);

FIG. 3 shows the results of oscillatory stress sweep experiments on a synthetic bone substitute in accordance with the invention;

FIG. 4 shows the results of the experiments depicted in FIG. 3 expressed as a function of shear strain;

FIG. 5 shows the results of oscillatory temperature sweep experiments on a synthetic bone substitute in accordance with the invention; and

FIG. 6 illustrates the results of viscosity/shear stress experiments conducted on a synthetic bone substitute in accordance with the invention.

FIG. 7 shows the particle size distribution of a synthetic bone substitute of the present invention.

FIG. 8 shows a microCT image of an extruded sample of the invention, enabling visualisation of the denser, lighter-coloured granules suspended in the hydrogel.

FIG. 9 shows a schematic of defect sectioning as carried out in example 5.

FIG. 10 shows histology slides stained using Sanderson's Rapid Bone Stain at 4 weeks at ×20 magnification. (a) βGran predicate control. (b) synthetic bone substitute of the present invention (βGel test material).

### DESCRIPTION

### 1. Preparation of a Synthetic Bone Substitute

A synthetic bone substitute in accordance with the invention was prepared by suspending β-tricalcium phosphate granules in a poloxamer hydrogel carrier. The β-tricalcium phosphate granules were previously sieved to provide two populations of granules having different average particle sizes prior to suspension. Techniques for sieving are described in, for example, US 2006/0110357.

In one specific example of preparing the synthetic bone substitute, the following steps were carried out to make the hydrogel carrier:

214.5 g Lutrol F127 microbeads were weighed into a mixing vessel;

500 g sterile water at 5° C. was poured onto the Lutrol microbeads and the two stirred together to dissolve the beads;

The mixture was refrigerated for 2 hours, removed from the refrigerator and stirred and then returned to the refrigerator. This process was repeated and then the mixture was refrigerated overnight.

To produce the particles, the following steps were carried out:

3 kg of β-Gran oven dried material (available from Orthos Ltd, Technium Springboard, Llantarnam Park, Cwmbran NP44 3AW, United Kingdom) was broken down using a pestle and mortar;

The material was sieved through a 500 micron sieve and the recovered material was passed through a 250 micron sieve. The sieve fractions were retained;

165 g of each fraction of the sieved granules was placed in porcelain trays and loaded into an oven set to 1000° C. where it was sintered for 6 hours;

The sintered material was resieved using the same gauge sieves and then sintered for a second time at 1100° C.;

The sintered particles were then sieved again to break up any agglomerates.

To prepare the synthetic bone substitute, 1071.38 g of the granules prepared were added to the prepared hydrogel and the mixture stirred. The gel was then refrigerated overnight.

Subsequently the synthetic bone substitute was sterilised, for example by gamma irradiation or electron beam sterilisation using standard techniques. Alternatively, the synthetic bone substitute may be sterilised using ethylene oxide.

### 2. Characteristics of the Product

### Sample Preparation

### Scanning Electron Microscopy (SEM) Analysis

The physical characteristics of the synthetic bone substitute in accordance with the invention, prepared as described above, were determined. A comparison was made with an existing synthetic bone substitute sold under the name Actifuse®.

A sample of each synthetic bone substitute was weighed (1 g) and dissolved in 1000 ml of milli-Q water to separate the suspended particles from the carrier matrix. A sample of the sediment was then filtered and dried (at 37° C.) on a glass coverslip, which was sputter-coated with a thin gold layer for SEM analysis.

### Scanning Electron Microscopy

A Zeiss Supra SEM with the following imaging parameters was used to image the particles and to obtain values for the principal axes.
Analyzed signal: secondary electrons
Gun: EHT 2 kV and 10 kV
Working distance: 5 mm

### Results

### Particle Size Analysis

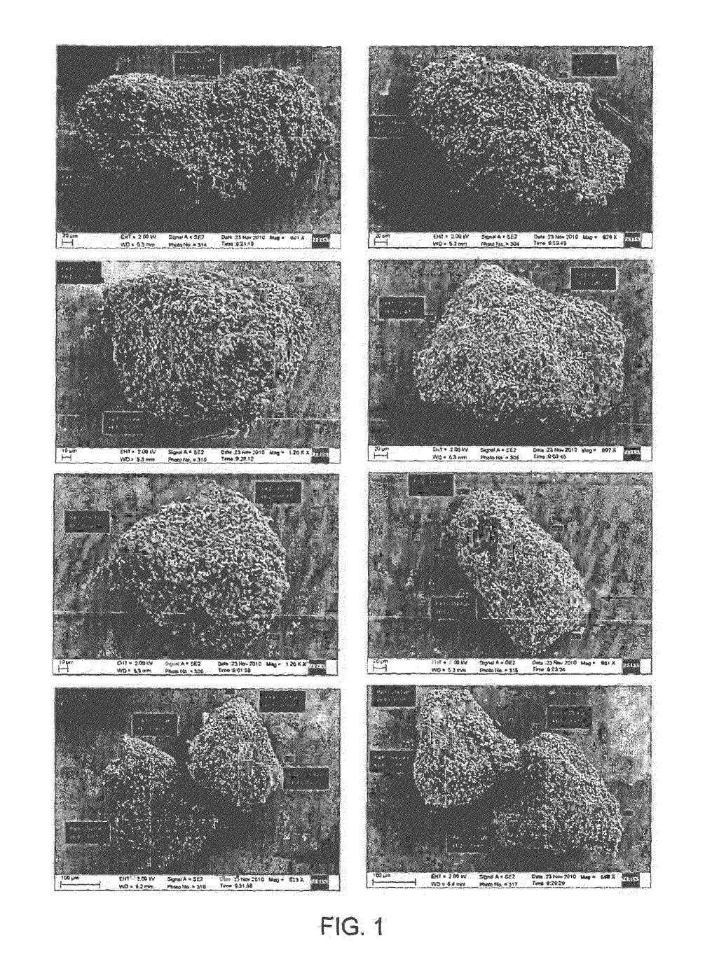

FIG. 1 represents the SEM images of the particles derived from a synthetic bone substitute in accordance with the invention. The shapes and sizes of the particles are irregular and variable. An estimate of the principal axes of the 2D images as well as a measure of particle size is given in Table 1. The arrows in the images (FIG. 1) indicate regions where the particles may have fractured during sample preparation or manufacture.

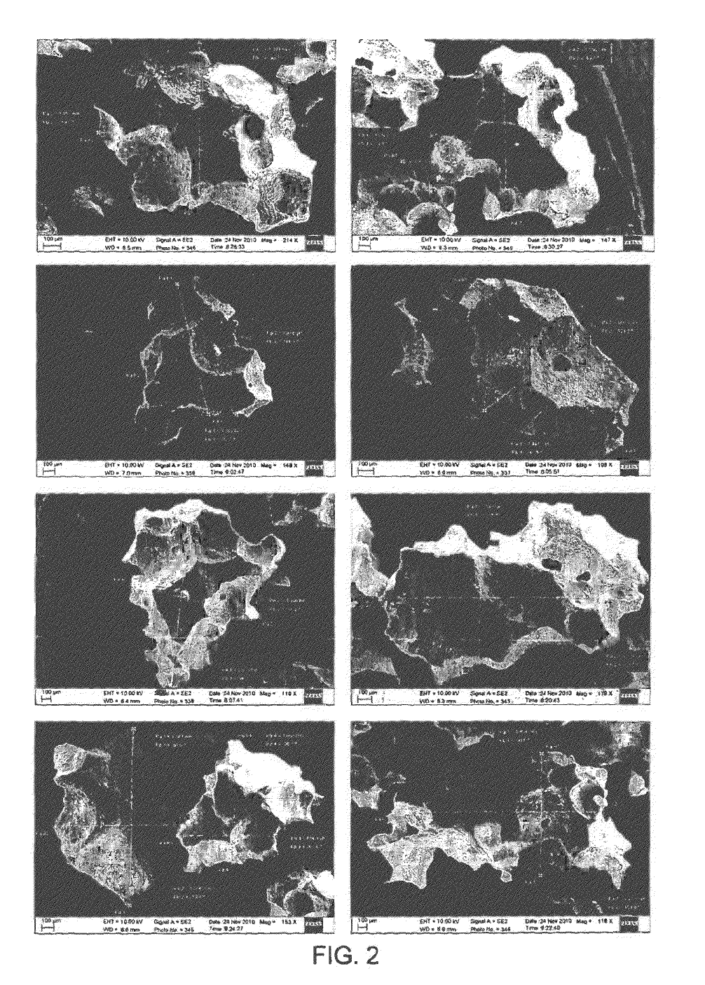

FIG. 2 shows micrographs of Actifuse® particles. The dimensions are again listed in Table 1. The particles were far bigger; more jagged and had larger pores in comparison to the particles in the synthetic bone substitute in accordance with the invention.

TABLE 1
Particle sizes obtained by SEM analysis

| | Synthetic bone substitute of | |
|---|---|---|
| | the invention (μm) | Actifuse (μm) |
| Principal axis (max) | 301.1 ± 77.3 | 1520.7 ± 378.4 |
| Principal axis (min) | 211.9 ± 42.8 | 1128.3 ± 287.2 |

### 3. Evaluation of the Handling Characteristics of a Synthetic Bone Substitute in Accordance with the Invention

Post-irradiation samples of a synthetic bone substitute of the invention comprising different poloxamer concentrations were evaluated by an experienced surgeon panel. The panel was asked to consider the handling characteristics of the material as they applied it in simulated fracture and osteotomy defects created in Sawbones® models and as they filled spinal interbody fusion devices.

A panel of experienced surgeon users was assembled. Each panel member had previously used at least one known synthetic bone substitute on multiple occasions clinically. Each panel member was supplied with two samples of synthetic bone substitute in accordance with the invention from each of the test batches containing sufficient material for several applications and asked to evaluate and score the performance of each sample when applying them manually into a simulated tibial defect created in a Sawbones® tibia model or when filling a spinal interbody fusion device. The samples were marked anonymously to blind the panelist from the composition of the sample being applied.

### Method

Four sample batches were prepared by suspending a mixture of β-tricalcium phosphate granules having first and second average particle sizes, the first average particle size being less than 250 μm and the second average particle size being about 250-500 μm, as described above, in a poloxamer carrier. The hydrogel concentration of each batch was modified to achieve final concentrations by weight of 25, 30, 40 & 45% w/w.

Samples were packed in a modified open-ended 10 ml polycarbonate syringe and sealed in a foil inner pouch and a paper/film outer pouch prior to irradiation. All samples were marked anonymously, bearing only a sample reference number and a bar-coded identification mark.

The samples were irradiated with gamma irradiation (Isotron plc) using a standard 25-35 kGy production cycle based on the anticipated sterilisation protocol where this is the normal cycle dose the product will receive (certificate of irradiation 0319560). Once the samples were returned from sterilisation they were placed into quarantine and stored at between 10° C. and 30° C. Samples were held in quarantine for 30 days post manufacture before release for testing.

Two defects were produced in a Sawbones® foam cortical shell tibia model (Ref 1117-20—Sawbones Europe AB., Malmo, Sweden). The first defect simulates a classic mid shaft fracture, the second simulating a high tibial wedge osteotomy. Two 13 mm “Saber” posterior lumbar interbody cage fusion devices (Ref 1874-250-09—DePuy Spine, Leeds) were also provided to simulate the spinal use of the synthetic bone substitute product

### Scaling

Each panel member was provided with two samples randomly selected from each of the prepared batches. They were asked to evaluate the performance of the handling characteristics by applying them in the simulated defects created in a Sawbones® model and by filling a spinal interbody fusion device, and then to score the performance subjectively using the following scale; Unacceptable—1, Acceptable—2, or Preferred—3.

### Conclusion

Several conclusions were immediately obvious from the exercise. The panel members were unanimous in that the lower 25% concentration didn't perform sufficiently in the manual application test and similarly that the higher 45% concentration proved too stiff to inject adequately. Overall the 30% w/w concentration material performed best in both application modes. It was observed that the higher 40% w/w concentration performed well in filling interbody fusion cages.

### 4. Rheology Testing

The synthetic bone substitute of the invention is better described as a soft-solid rather than a liquid, and, as such, solid characteristics such as rigidity and shear strength provide a relevant description of “physical” properties. The test methods employed for characterising the synthetic bone substitute focus, therefore, on quantifying its soft-solid properties.

Complex modulus (G*): The ratio of shear stress to shear strain—a measure of the shear rigidity of the sample. Measured in Pascals.

Yield Stress: The stress required to disrupt elastic soft solid structure and elicit viscous/plastic flow. Yield stress is expected to show a close correlation to handling characteristics, notably the ease with which the product can be syringed and “worked” by the surgeon.

Yield Strain: The deformation at the yield point. Yield strain may prove a key characteristic, a higher yield strain lending a stretchy toughness to a sample, whilst a low yield strain is more likely to result in a crumbly, brittle “cheesier” texture.

Zero-shear viscosity: Viscosity/stress or viscosity/shear rate profiles often exhibit a plateau of Newtonian behaviour (constant viscosity) at very low stresses and low shear rates. The viscosity in this region is known as the zero-shear viscosity and can be thought of as the viscosity “at rest” or under very slow creeping-flow conditions.

### Equipment

All testing was performed on a research rheometer (AR2000, TA Instruments Ltd). A 40 mm diameter plate-plate system with a sample gap of 1.5 mm was used for all the testing. Crosshatched versions of the plates were employed to eliminate any wall-slip effects likely to be seen when testing solid suspensions with smooth-surfaced plates and therefore to promote shear through the bulk of the sample. A solvent trap cover was employed to minimize any drying effects.

However, due to the large mass and subsequent large heat capacity of these accessories, a significant temperature offset exists between the measured temperature and the actual sample temperature. To remedy this situation a “span and offset” calibration was performed: a sample of the synthetic bone substitute was loaded onto the rheometer and a temperature probe was pushed into the sample. The required temperature was then set to 10° C., 20° C. and 40° C. and, following temperature equilibration, the actual temperature was recorded.

### Test Methods

Three test methods were employed:

1. Oscillatory stress sweep: To obtain the complex modulus, yield stress and yield strain
2. Oscillatory temperature sweep: To obtain the complex modulus as a function of temperature
3. Viscosity/shear stress profile: To obtain a zero-shear/creep viscosity at body temperature.

### Oscillatory Testing Methods

In an oscillatory test, small, sinusoidal rotational (clockwise then counter-clockwise) stresses or strains (depending on whether a controlled stress or controlled strain mode of test is employed) are applied to the sample and its response is observed. From this, a knowledge of the material's resistance to deformation (complex modulus, G*) and elasticity (phase angle, δ) can be obtained. Stress, strain, temperature or frequency of oscillation can be varied and the resulting change in viscoelastic properties monitored.

### Oscillatory Stress Sweep

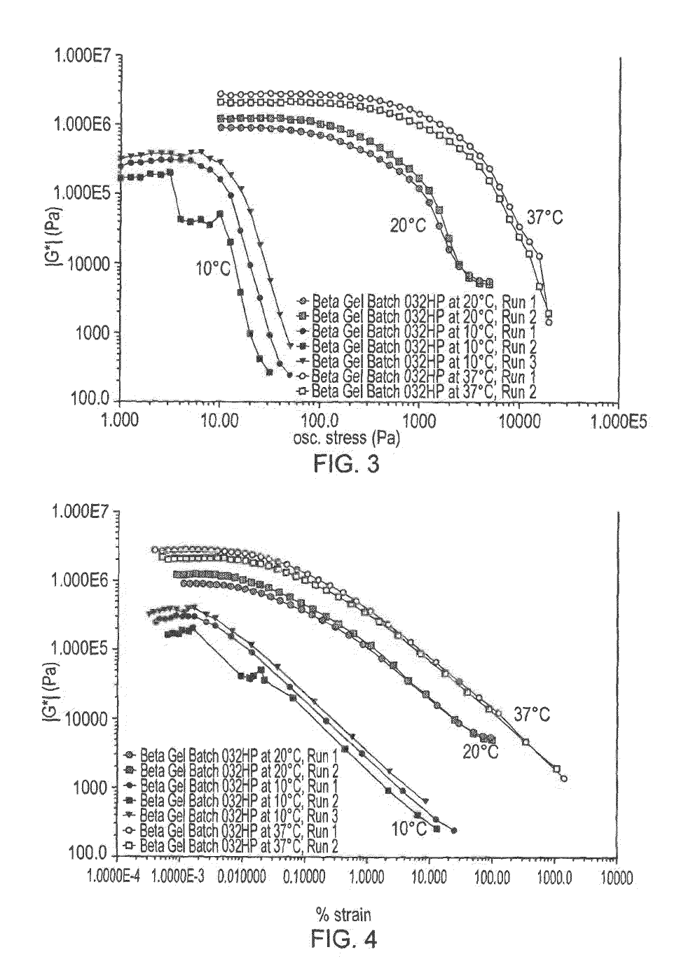

In the oscillatory stress sweep the applied stresses are incremented until the sample undergoes a structural yield. Results of the testing on β-Gel are shown in FIG. 3.

### Comments

All three samples show a distinct yielding with modulus, decreasing by several decades.
Due to the erratic result produced for run 2 at 10° C. a third run was performed.
The plateau moduli and the stresses over which the yields occur vary significantly with temperature, with values increasing with increasing temperature.
In order to obtain a quantified yield stress value for comparative purposes the stress required to elicit a 90% decrease in modulus from the plateau value was interpolated.

Approximate values are given in the table below:

| | 10° C. | 10° C. | 20° C. | 20° C. | 37° C. | 37° C. |
|---|---|---|---|---|---|---|
| | Run 1 | Run 3 | Run 1 | Run 2 | Run 1 | Run 2 |
| Complex Modulus | 3.00E+05 | 3.71E+05 | 8.90E+05 | 1.23E+06 | 2.72E+06 | 2.10E+06 |
| Plateau (Pa) | | | | | | |
| “Yield threshold” | 3.00E+04 | 3.71E+04 | 8.90£+04 | 1.23E+05 | 2.72E+05 | 2.10E+05 |
| (10% of complex | | | | | | |
| modulus (Pa)) | | | | | | |
| Interpolated yield | 15 | 21 | 1200 | 1300 | 4300 | 4100 |
| stress (Pa) | | | | | | |

### Strain Responses

By re-plotting the results as a function of shear strain it is possible to gain an insight into the deformability of the product. The results are depicted in FIG. 4.

Qualitatively, it can be seen that the sample starts to yield at a lower strain at 10° C. than at 20° C. and 37° C. The strain values associated with the 90% yields quantified above are as follows:

| | 10° C. | 10° C. | 20° C. | 20° C. | 37° C. | 37° C. |
|---|---|---|---|---|---|---|
| | Run 1 | Run 3 | Run 1 | Run 2 | Run 1 | Run 2 |
| Strain at 90% | 0.06 | 0.07 | 1.8 | 1.6 | 1.3 | 1.6 |
| yield stress | | | | | | |
| (%) | | | | | | |

### Oscillatory Temperature Sweeps

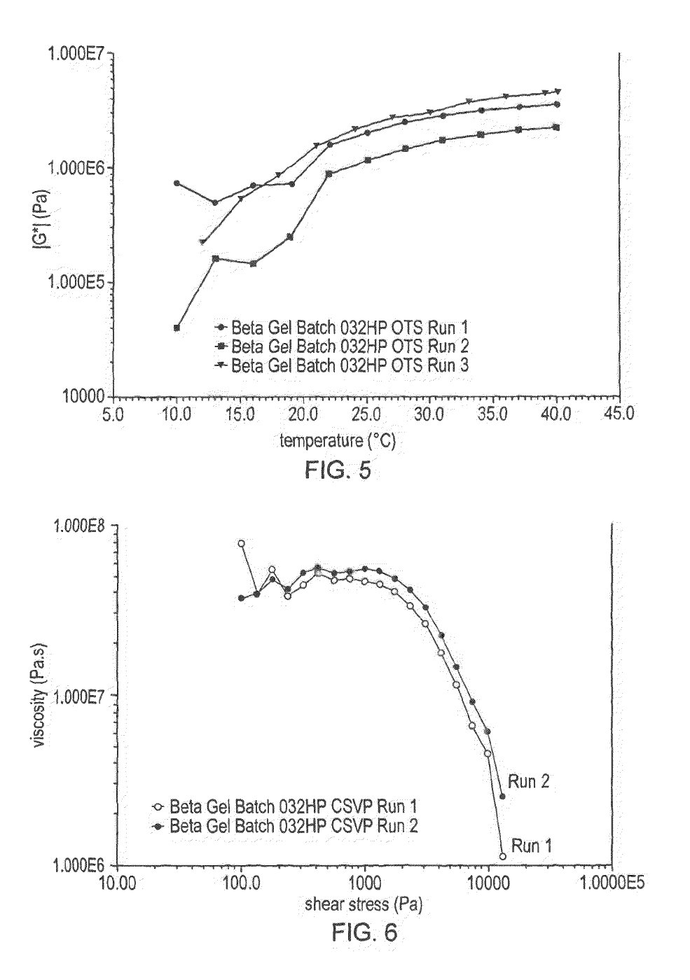

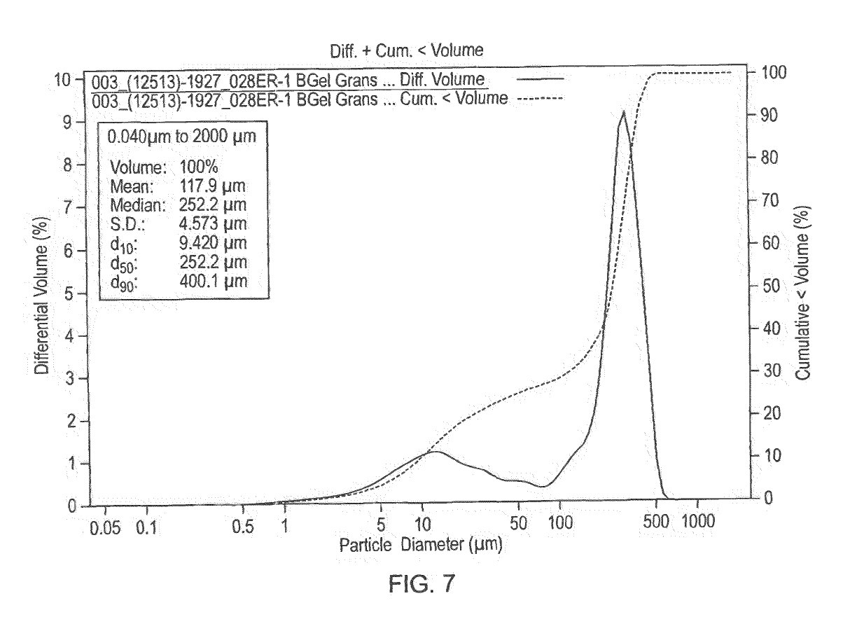

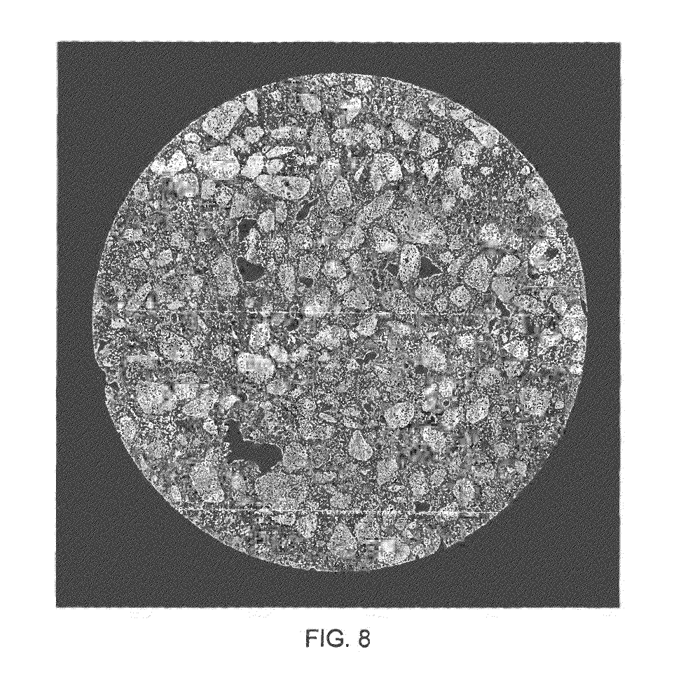

In the oscillatory temperature sweep the sample is oscillated at a single low applied strain whilst temperature is swept. The results of the oscillatory temperature sweep are shown in FIG. 5.

### Comments

A modulus increase is observed across the temperature range 10° C. to 40° C.
Results at lower temperatures can be erratic.
A significant difference between run 1 and 2 prompted a third run, showing a close agreement with run 1.

### Viscosity/Shear Stress Profile

In the shear stress sweep an incrementing shear stress (in one direction, in contrast to the oscillatory stress sweep) is applied to the sample and the resulting deformation rate (shear rate) is monitored, from which viscosity is calculated at each shear stress. The results shown in FIG. 6 were obtained at 37° C.

### Comments:

The Newtonian plateau can be clearly seen at low stresses.
Estimated zero-shear viscosities are:

### Run 1: 4.83×10⁷ Pa·s

### Run 2: 5.57×10⁷ Pa·s

### 5. Histological and Resorption Analysis

### Introduction

A synthetic bone substitute of the present invention (hereafter; βGel) comprising beta tricalcium phosphate (βTCP) in a reverse phase hydrogel carrier (Table 2) was prepared to a) determine the efficacy of βGel as a bone void filler; b) evaluate its resorption behaviour in vivo, and; c) study and detect any adverse tissue reaction that may occur while the βGel is resorbed

TABLE 2
Summary of βGel composition.

| Parameter | Comment |
|---|---|
| Granule composition (ASTM F1088) | βTCP [Ca₃(PO₄)₂] ≧95%; HA |
| | balance [Ca₁₀(PO₄)₆(OH)₂] |
| Granule size distribution | Bi-modal (5-100 μm; 100-500 μm) |
| Carrier composition, water:poloxamer | 70:30 |
| (wt/wt) | |
| Granule:hydrogelcarrier (vol/vol) | 60:40 |

βGel is designed to have excellent handling and biological properties. The particles of βGel are identical in chemical composition to that of βGran (Orthos; Table 3), which was used as a predicate control in the present study and has proven safe and effective clinical performance. βGran particles are of a similar size to that of other commercially available synthetic osteoconductive scaffolds.

TABLE 3
Summary of βGran used in the present study.

| Parameter | Comment |
|---|---|
| Granule composition (ASTM F1088) | βTCP [Ca₃(PO₄)₂] ≧95%; HA |
| | balance [Ca₁₀(PO₄)₆(OH)₂] |
| Granule size distribution | 1-2 mm |

In βGel, smaller granules of βGran are mixed with a biocompatible hydrogel carrier (a poloxamer). In a previous in vivo study in sheep the βGran synthetic osteoconductive scaffold, loaded with autologus bone marrow, resulted in the production of healthy bone throughout surgically created defects. Close adaption and an intimacy between the bone and implant concurrent with progressive resorption of the scaffold occurred. No adverse foreign body responses were observed.

The particle size distribution of βGel contains a fraction (<30%) of particles smaller than 100 μm. It was important therefore to assess its functional biocompatibility and in particular the inflammatory response to the particles.

### Materials and Methods

Three groups of test subjects were investigated (Table 4). Eleven New Zealand White rabbits of at least 3.0 kg at the start of the test were utilised for each in-life group. In addition ten cadavers were used to establish a baseline for resorption quantification. Critical size defects (6 mm diameter, 10 mm depth), and were created in the lateral condyles of both left and right legs using a low speed drill and extensive irrigation to minimise bone necrosis. Each defect was filled with 0.125 mL βGel (left condyle) and 0.15 mL βGran (right condyle) mixed with autologous surgical site blood, and sealed with bone wax.

TABLE 4
Summary of implantation sites for βGel and βGran in
a rabbit femoral defect model.

| | | Number of Implant Sites | |
|---|---|---|---|
| | Number | βGel (left femoral | βGran (right femoral |
| Duration | of Animals | condyle) | condyle) |
| 0 weeks | 10 (cadavers) | 10 | 10 |
| 4 weeks | 11 | 11 | 11 |
| 8 weeks | 11 | 11 | 11 |
| 12 weeks | 11 | 11 | 11 |

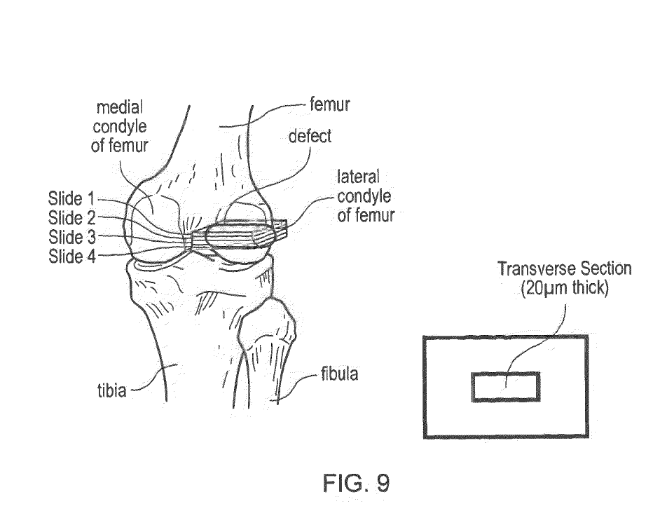

Post-operative and post-termination radiographs were collected. Macroscopic observations were documented at the time of implant site exposure after termination. The explanted tissue was processed using standard histological techniques. Four sections through each condylar defect were prepared for histological examination (FIG. 9). Following processing three slides per defect were stained with Sanderson's Rapid Bone Stain, and one with Mason's Trichrome. All sections were analysed by a veterinary pathologist to assess product resorption relative to the baseline cadaver controls.

The regional draining lymph nodes (inguinal) were also assessed for any gross lesions and photos were taken. At least one draining lymph node per rabbit was harvested and fixed in 10% NBF for histopathology processing. If an abnormality was observed grossly, both lymph nodes were collected. If the inguinal draining lymph nodes were not identified grossly, the tissue in the general area of the inguinal lymph node location was collected and/or other draining lymph nodes were harvested.

The measurement of bone formation captured the amount of new lamellar bone (excluding bone marrow) within the implant site. The tissue reaction ingrowth into the device captured the new lamellar bone, fibrosis and inflammatory cells found surrounding and separating the particles of the implant materials.

### Results

### Tissue Reaction

### Macroscopic Observations

Macroscopic observations at all time points were similar and none of the findings appeared to be treatment-related. At four weeks n=4 draining lymph nodes from the βGel implantation sites and n=3 draining lymph nodes from the βGran implantation sites appeared grossly increased in size. Microscopic evaluation of this finding appeared to be a normal immune response to environment, and not a response to the implant materials.

### Microscopic Observations

4 Week and 8 Weeks

For both implantation materials, admixed with the fibrosis and inflammatory reaction, was a minimal to moderate amount of neovascularisation. The tissue reaction of all of the βGel implant sites contained a mild to marked amount of macrophages and a minimal to mild amount of multinucleated giant cells. The tissue reaction of most of the βGel implant sites also had a minimal to mild amount of lymphocytes. Similar microscopic observations were recorded for the βGel and βGran implant sites at both 4 and 8 weeks.

12 Weeks

The tissue reaction of both the βGel and βGran implantation sites contained minimal to moderate amount of macrophages, a minimal to mild amount of multinucleated giant cells, and a minimal amount of lymphocyctes. There was a minimal to mild amount of neovascularisation observed for both materials. There were no microscopic changes in any of the lymph nodes examined at 12 weeks.

### Bone Formation

4 and 8 Weeks

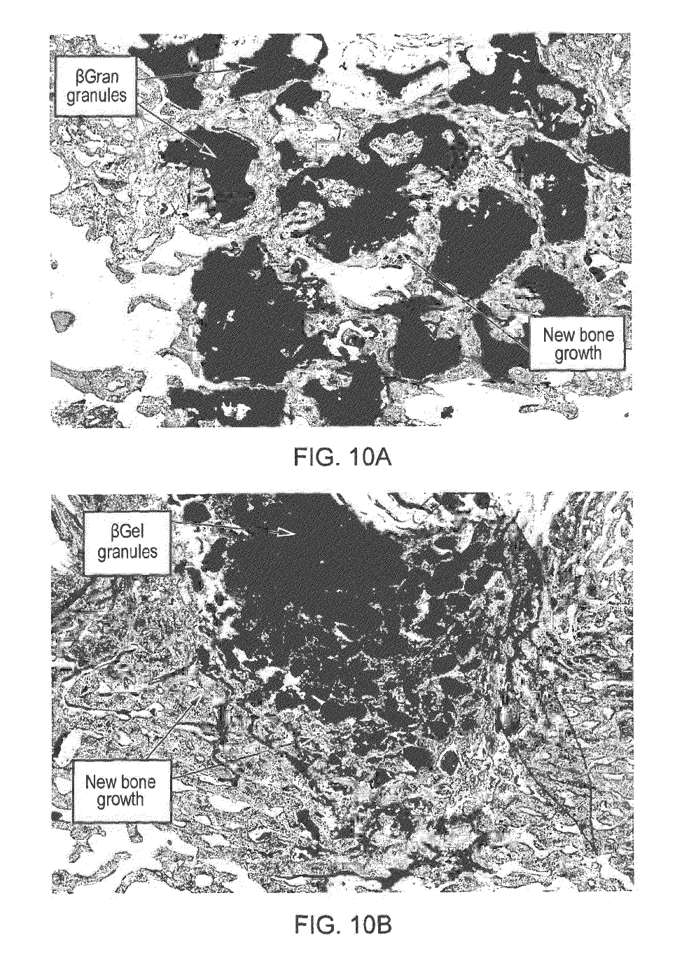

Minimal to marked amount of mature lamellar bone were observed at both time points for both material implantation sites (FIG. 10, showing histology slides stained using Sanderson's Rapid Bone Stain at 4 weeks at ×20 magnification. (a) βGran predicate control. (b) βGel test material; Table 5).

12 Weeks

Minimal to marked amount of mature lamellar bone were observed in both βGel and βGran implantation sites (Table 5).

### Implant Resorption

4 and 8 Weeks

At 4 weeks the rate βGel granule resorption was 2.6-times greater than that of the βGran predicate article; by 8 weeks the rate of resorption was 1.5-times greater than the predicate (Table 5).

12 Weeks

The rate of βGel granule resorption at 12 weeks was 1.5-times greater than βGran.

TABLE 5
Summary of the semi-quantitative data for implant resorption
and remodelling with respect to time in vivo.

| | | 4 weeks | 8 weeks | 12 weeks |
|---|---|---|---|---|
| Neovasularisation | βGel | 2 | 2 | 1 |
| score^{a} | βGran | 1 | 1 | 1 |
| Bone formation score^{b} | βGel | 2 | 3 | 2 |
| | βGran | 2 | 3 | 3 |
| Implant resorption^{c} | βGel | 41% | 67% | 93% |
| | βGran | 16% | 45% | 62% |

^{a}0 = absent; 1 = minimal/slight (minimal capillary proliferation, (focal, 1-3 capillary buds), or small blood vessels (venules, and/or arterioles)); 2 = mild (groups of 4-7 capillaries with supporting fibroblastic structures); 3 = moderate (broad band of capillaries with supporting structures); 4 = marked (extensive band of capillaries with supporting fibroblastic structures).
^{b}0 = absent; 1 = minimal/slight (>0 up to 25% of the implant field); 2 = mild (>25 up to 50% of the implant field); 3 = moderate (>50% up to 75% of the implant field); 4 = marked/severe (>75 up to 100% of the implant field).
^{c}Calculated relative to the 0 week cadaver control sites.

### 4. Conclusion

Over a 12 week implantation period the tissue reactions of both the βGel and βGran implantation sites were similar, with similar immunological responses identified during histological examination. The materials resulted in a similar amount of mature lamellar bone formation at each time point, whereas the βGel material resorbed at a greater rate compared to the predicate, βGran.

Based on the data obtained at 4, 8 and 12 weeks the tissue response and bone formation of a novel bone graft substitute material, βGel, was equivalent to that of a predicate material, βGran.

### 6. Effect of Particle Size on Flow Properties

### Test Method

Injectability tests were carried out at a loading rate of 15 mm/min, a temperature of 20° C. and using 40:60 (hydrogel:particle) synthetic bone substitutes produced using the particle size ranges detailed in Table 2. They were produced by sieving samples from a single batch of β-tricalcium phosphate using titanium sieves and a table top sieve shaker for 15 min.

Particle size analyses were also carried out for each particle size range to assess whether the means and medians were indeed comparable.

TABLE 2
Test materials particle ranges

| Batch Number | Particle Size Range (μm) |
|---|---|
| 050KP | Unsieved (nominal 250-500 range) |
| 050KP | 200-500 |
| 050KP | 300-400 |

### Results & Discussion

As shown in Table 3 below, the force required to extrude the material increased with each reduction in particle size range, but this can only be shown to be statistically significant (p<0.05) when comparing the two sieved samples.

TABLE 3
Results of injectability tests using different particle size ranges,
carried out at a rate of 15 mm/min

| Range | Average Force (N) | S.D. |
|---|---|---|
| Unsieved (nominal 250-500 range) | 46 | 12 |
| 200-500 μm | 56 | 8 |
| 300-400 μm | 93 | 13 |

This suggested relationship between particle size range and injectability indicates that there may be an optimal range in terms of handling

1-29. (canceled)

30. A synthetic bone substitute, comprising a mixture of osteoconductive particles of first and second average particle sizes, suspended in a water-soluble reverse-phase hydrogel carrier in which the first average particle size is less than 100, and the second average particle size is 100-500 μm.

31. A synthetic bone substitute according to claim 30, in which the first average particle size 1-50 μm and the second average particle size is about 125-450 μm.

32. A synthetic bone substitute according to claim 30, in which the hydrogel is a poloxamer.

33. A synthetic bone substitute according to claim 30, in which the synthetic bone substitute comprises the hydrogel carrier at a weight to weight ratio of between 25:75 to 35:65 with water.

34. A synthetic bone substitute according to claim 30, wherein the osteoconductive particles and hydrogel carrier are present in a volume:volume ratio of between 70:30 and 50:50.

35. A synthetic bone substitute according to claim 30, wherein the osteoconductive particles are tricalcium phosphate particles.

36. A synthetic bone substitute according to claim 30, including a radio opaque material; a component which increases the visibility of the synthetic bone substitute in use; bone powder, a growth factor, bone morphogenic protein, gypsum, hydroxyapatite, other calcium phosphate, carbonate or sulphate, or a combination thereof.

37. A kit comprising a packaging and/or delivery device and a synthetic bone substitute in accordance with claim 30.

38. A kit according to claim 37, in which the packaging is sterile.

39. A kit according to claim 38 for single or multiple use.

40. A kit according to claim 37 in which the delivery device is a syringe suitable for administering synthetic bone substitute to repair a bone defect or to fill an implant.

41. A method of producing a synthetic bone substitute, the method comprising providing a mixture of osteoconductive particles of first and second average particle sizes, in which the first average particle size is less than 100 μm and the second average particle size is 100-500 μm, and suspending the particles in a reverse-phase hydrogel carrier.

42. A method according to claim 41, wherein the first average particle size is about 1-50 μm and the second average particle size is 125-450 μm,

43. A method according to claim 41, wherein the osteoconductive particles are tricalcium phosphate granules.

44. A method according to claim 41 in which the mixture of osteoconductive granules having the first and second average particle sizes is provided by sieving a mixture of tricalcium phosphate granules.

45. A method according to claim 41 in which the mixture of osteoconductive particles and hydrogel carrier comprises the hydrogel carrier at a weight to weight ratio of between 25:75 to 35:65 with water.

46. A method according to claim 41 wherein the osteoconductive particles and hydrogel carrier are present in a volume:volume ratio of between 70:30 and 50:50.

47. A method according to claim 41 in which the hydrogel is a poloxamer.

48. A synthetic bone implant comprising a synthetic bone substitute according to claim 30.

49. An implant according to claim 48, which is shaped to fill a bone defect.

50. A method of repairing a bone defect, comprising introducing a synthetic bone substitute according to claim 30 into the bone defect, and allowing the synthetic bone substitute to set.

51. A method according to claim 50 in which the bone defect is naturally occurring or artificially generated.
